(19) United States
(12) Patent Application Publication
(10) Pub. No.: US 2025/0285281 A1
(43) Pub. Date: Sep. 11, 2025

(54) METHODS AND SYSTEMS FOR MEDICAL IMAGING

(71) Applicant: SHANGHAI UNITED IMAGING HEALTHCARE CO., LTD., Shanghai (CN)

(72) Inventors: Jiansen LI, Shanghai (CN); Bing LIU, Shanghai (CN); Xuan WANG, Shanghai (CN)

(73) Assignee: SHANGHAI UNITED IMAGING HEALTHCARE CO., LTD., Shanghai (CN)

(21) Appl. No.: 19/218,348

(22) Filed: May 25, 2025

Related U.S. Application Data
(63) Continuation of application No. PCT/CN2024/102455, filed on Jun. 28, 2024.

(30) Foreign Application Priority Data
Jun. 30, 2023 (CN) 202310799996.6

(51) Int. Cl. G06T 7/00; G06T 7/215; G06T 11/00; G06T 11/20

(52) U.S. Cl. CPC G06T 7/0016; G06T 7/215; G06T 11/008; G06T 11/206; G06T 2207/10088; G06T 2207/30048; G06T 2207/30056; G06T 2210/41; G06T 2211/464

(57) ABSTRACT

Systems and methods for medical imaging are provided. The systems obtain motion state information of a region of interest (ROI) of a target object during a scanning process of the target object. The scanning process includes multiple scan phases that are scanned sequentially. The motion state information includes at least one of a movement probability or a movement level representing a position change of the target object in each scan phase. The systems perform at least one of during the scanning process, displaying the motion state information corresponding to at least one of multiple scan phases that has been scanned, or after the scanning process is completed, displaying the motion state information corresponding to the multiple scan phases and a medical image of the target object obtained in the scanning process.

### CROSS-REFERENCE TO RELATED APPLICATIONS

This application is a continuation of International Application No. PCT/CN2024/102455, filed on Jun. 28, 2024, which claims priority to Chinese Patent Application No. 202310799996.6, filed on Jun. 30, 2023, the entire contents of which are hereby incorporated herein by reference.

### TECHNICAL FIELD

The present disclosure relates to the field of medical technology, and in particular, to methods and systems for medical imaging.

### BACKGROUND

In a scanning process, it is often necessary for a scanned object to maintain a certain static state to prevent motion artifact(s) on an image obtained during the scanning process, thereby improving the imaging effect. However, it is difficult to know if and how much the scanned object moves during the scanning process. Usually, after the scanning process is completed, the scanned object is rescanned when the motion of the scanned object causes serious image artifacts, which wastes a lot of time and affects imaging efficiency. Therefore, it is desirable to provide methods and systems for medical imaging to display the movement of the scanned object.

### SUMMARY

An aspect of the present disclosure relates to a method for medical imaging. The method is implemented on a medical imaging device including at least one processor and at least one storage device. The method includes obtaining motion state information of a region of interest (ROI) of a target object during a scanning process of the target object. The scanning process includes multiple scan phases that are scanned sequentially. The motion state information includes at least one of a movement probability or a movement level representing a position change of the target object in each scan phase. The method further includes performing at least one of during the scanning process, displaying the motion state information corresponding to at least one of multiple scan phases that has been scanned, or after the scanning process is completed, displaying the motion state information corresponding to the multiple scan phases and a medical image of the target object obtained in the scanning process.

In some embodiments, during the scanning process, the displaying the motion state information corresponding to at least one of multiple scan phases that has been scanned includes forming, based on an execution order of the at least one of multiple scan phases, a first visual chart of the motion state information corresponding to the at least one of multiple scan phases that has been scanned; and displaying the first visual chart.

In some embodiments, the displaying the first visual chart includes obtaining a preset threshold; and highlighting elements of the first visual chart corresponding to the motion state information based on the preset threshold.

In some embodiments, after the scanning process is completed, the displaying the motion state information corresponding to the multiple scan phases and a medical image of the target object obtained in the scanning process includes displaying, based on a preset display rule in a same display interface, the motion state information corresponding to the multiple scan phases and the medical image of the target object.

In some embodiments, the preset display rule includes at least one of: displaying the motion state information corresponding to the multiple scan phases in a displaying region on the display interface; displaying the motion state information exceeding a preset threshold in the displaying region; forming, based on an execution order of the multiple scan phases, a second visual chart of the motion state information corresponding to the multiple scan phases, and displaying the second visual chart in the displaying region; displaying the second visual chart in the displaying region, and highlighting elements of the second visual chart corresponding to the motion state information exceeding the preset threshold; or marking an image region corresponding to the motion state information exceeding the preset threshold in the medical image.

In some embodiments, the displaying region includes a region, in the display interface, not occupied by the medical image, at least one of four corners of the medical image, or a pop-up window of the display interface.

In some embodiments, the first visual chart or the second visual chart includes at least one of a scatter chart, a line chart, or a bar chart.

In some embodiments, the preset threshold is related to a scan site of the ROI of the target object.

In some embodiments, the method further includes storing, based on a preset storage rule, the medical image of the target object and the motion state information corresponding to the multiple scan phases.

In some embodiments, the preset storage rule includes storing, in a first preset region of the at least one storage device, the medical image of the target object, the motion state information corresponding to the multiple scan phases, and a second visual chart of the motion state information corresponding to the multiple scan phases.

In some embodiments, the preset storage rule includes storing the medical image of the target object in a first preset region of the at least one storage device, and storing the motion state information corresponding to the multiple scan phases and a second visual chart of the motion state information corresponding to the multiple scan phases in a second preset region of the at least one storage device.

In some embodiments, the preset storage rule includes storing, in a same preset region or different preset regions of the at least one storage device, motion state information corresponding to the multiple scan phases and second visual charts obtained in different scanning processes.

In some embodiments, the obtaining motion state information includes obtaining the motion state information by scanning the target object using a motion detection device inserted into the multiple scan phases in the scanning process.

Another aspect of the present disclosure relates to a system for medical imaging. The system includes at least one storage device including a set of instructions and at least one processor in communication with the at least one storage device. When executing the set of instructions, the at least one processor is directed to cause the system to implement operations. The operations include obtaining motion state information of a region of interest (ROI) of a target object during a scanning process of the target object. The scanning process includes multiple scan phases that are scanned sequentially. The motion state information includes at least one of a movement probability or a movement level representing a position change of the target object in each scan phase. The operations further include performing at least one of during the scanning process, displaying the motion state information corresponding to at least one of multiple scan phases that has been scanned, or after the scanning process is completed, displaying the motion state information corresponding to the multiple scan phases and a medical image of the target object obtained in the scanning process.

A further aspect of the present disclosure relates to a non-transitory computer readable medium including executable instructions. When the executable instructions are executed by at least one processor, the executable instructions direct the at least one processor to perform a method. The method includes obtaining motion state information of a region of interest (ROI) of a target object during a scanning process of the target object. The scanning process includes multiple scan phases that are scanned sequentially. The motion state information includes at least one of a movement probability or a movement level representing a position change of the target object in each scan phase. The method further includes performing at least one of during the scanning process, displaying the motion state information corresponding to at least one of multiple scan phases that has been scanned, or after the scanning process is completed, displaying the motion state information corresponding to the multiple scan phases and a medical image of the target object obtained in the scanning process.

A still further aspect of the present disclosure relates to a method for medical imaging. The method is implemented on a medical imaging device including at least one processor and at least one storage device. The method includes obtaining a scanning sequence for scanning a target object, the scanning sequence including multiple scan phases; for at least one of the multiple scan phases, obtaining motion monitoring signals of the target object by applying a motion detection sequence before the scan phase; generating motion state information of the target object based on the motion monitoring signals, the motion state information including a movement probability or a movement level representing a position change of the target object in the scan phase; and controlling a display interface of the medical imaging device to display the movement probability or the movement level.

In some embodiments, the display interface sequentially displays movement probabilities or movement levels corresponding to the multiple scan phases in a form of a scatter plot, a line chart, or a bar chart.

In some embodiments, the method further inlcudes generating a motion projection of the target object based on the motion monitoring signals, the motion projection being a one-dimensional projection or a multi-dimensional projection of the motion monitoring signals; generating movement probability based on the motion projection of the target object and reference information.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary medical imaging system according to some embodiments of the present disclosure;

FIG. 2 is a block diagram illustrating an exemplary processing device according to some embodiments of the present disclosure;

FIG. 3 is a flowchart illustrating an exemplary process for medical imaging according to some embodiments of the present disclosure;

FIG. 4 is a schematic diagram illustrating imaging sub-sequences and auxiliary sub-sequences according to some embodiments of the present disclosure;

FIGS. 5A-5C are schematic diagrams each of which illustrates an exemplary first visual chart according to some embodiments of the present disclosure;

FIGS. 6A-6C are schematic diagrams each of which illustrates an exemplary first visual chart with highlighting elements according to some embodiments of the present disclosure;

FIG. 6D is a schematic diagrams illustrating an exemplary first visual chart according to some embodiments of the present disclosure;

FIG. 6E is a schematic diagrams illustrating exemplary warning information according to some embodiments of the present disclosure;

FIGS. 7A-7B are schematic diagrams each of which illustrates an exemplary display interface according to some embodiments of the present disclosure;

FIG. 8 is a flowchart illustrating an exemplary process for medical imaging according to some embodiments of the present disclosure; and

FIGS. 9A-9C are schematic diagrams illustrating exemplary storing of medical images and motion state information according to some embodiments of the present disclosure.

### DETAILED DESCRIPTION

In the following detailed description, numerous specific details may be set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments may be readily apparent to those skilled in the art, and the general principles defined herein may be applied to other embodiments and applications without departing from the spirit and scope of the present disclosure. Thus, the present disclosure may be not limited to the embodiments shown, but to be accorded the widest scope consistent with the claims.

The terminology used herein may be for the purpose of describing particular example embodiments only and may be not intended to be limiting. As used herein, the singular forms “a,” “an,” and “the” may be intended to include the plural forms as well, unless the context clearly indicates otherwise. It may be understood that the terms “system,” “unit,” “module,” and/or “block” used herein are one method to distinguish different components, elements, parts, sections, or assemblies of different levels in ascending order. However, the terms may be displaced by another expression if they achieve the same purpose.

The modules (or units, blocks, units) described in the present disclosure may be implemented as software and/or hardware modules and may be stored in any type of non-transitory computer-readable medium or other storage devices. In some embodiments, a software module may be compiled and linked into an executable program. It may be appreciated that software modules may be callable from other modules or from themselves, and/or may be invoked in response to detected events or interrupts. Software modules configured for execution on computing devices may be provided on a computer-readable medium or as a digital download (and can be originally stored in a compressed or installable format that requires installation, decompression, or decryption prior to execution). Such software code may be stored, partially or fully, on a memory device of the executing computing device, for execution by the computing device. Software instructions may be embedded in a firmware, such as an EPROM. It may be further appreciated that hardware modules (e.g., circuits) may be included in connected or coupled logic units, such as gates and flip-flops, and/or may be included in programmable units, such as programmable gate arrays or processors. The modules or computing device functionality described herein may be preferably implemented as hardware modules, but may be software modules as well. In general, the modules described herein refer to logical modules that may be combined with other modules or divided into units despite their physical organization or storage.

Certain terminology has been used to describe embodiments of the present disclosure. For example, the terms “one embodiment,” “an embodiment,” and/or “some embodiments” may mean that a particular feature, structure, or characteristic described in connection with the embodiment is in at least one embodiment of the present disclosure. Therefore, it is emphasized and should be appreciated that two or more references to “an embodiment” or “one embodiment” or “an alternative embodiment” in various portions of this specification may not necessarily all referring to the same embodiment. Furthermore, the particular features, structures, or characteristics may be combined as suitable in one or more embodiments of the present disclosure.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings may be for the purpose of illustration and description only and may be not intended to limit the scope of the present disclosure.

The flowcharts used in the present disclosure may illustrate operations that systems implement according to some embodiments of the present disclosure. It is to be expressly understood, the operations of the flowcharts may be implemented not in order. Conversely, the operations may be implemented in inverted order, or simultaneously. Moreover, one or more other operations may be added to the flowcharts. One or more operations may be removed from the flowcharts.

Conventional medical imaging, such as magnetic resonance imaging (MRI), and especially 3D magnetic resonance scanning, are relatively slow. For high-resolution imaging, such as brain imaging, the scanning time is longer. The scanned object inevitably moves during the imaging process. After the scanned object moves, artifacts or image blur caused by the motion will appear on the images obtained by the medical imaging, thereby affecting the image quality, and accordingly affecting the diagnosis and treatment of the scanned object. In addition, for some medical imaging, it is not possible to intuitively distinguish image abnormalities caused by motion or disease on the generated images. For example, in spectroscopy and arterial spin labeling (ASL) imaging, motion and disease can cause abnormalities in spectral lines or abnormalities in ASL cerebral perfusion images.

The present disclosure provides systems and methods for medical imaging. The systems may obtain motion state information of a region of interest (ROI) of a target object during a scanning process of the target object, wherein the scanning process includes multiple scan phases that are scanned sequentially. Further, the systems may perform at least one of: during the scanning process, displaying the motion state information corresponding to at least one of multiple scan phases that has been scanned; or after the scanning process is completed, displaying the motion state information corresponding to the multiple scan phases and a medical image of the target object obtained in the scanning process.

According to the embodiments of the present disclosure, the motion state information of the target object may be displayed in real time during the scanning process, which can help to better control the scanning process without affecting the imaging effect of the medical imaging when the motion amplitude of the target object is relatively large. For example, when the motion amplitude of the target object is relatively large, the scanning process may be terminated in advance or the target object may be reminded in real time to reduce movement, which can avoid wasting scanning time, improve efficiency, and reduces the need for MRI-enhanced drug injection of the target object caused by secondary scanning due to serious artifacts caused by motion after image generation.

According to the embodiments of the present disclosure, after the scanning process is completed, the motion state information and the obtained medical image of the target object may be displayed, which makes it easier for a user (e.g., a doctor) to analyze the medical image. When artifacts caused by motion are small or do not affect lesion analysis, the user may make a diagnosis without repeating scans of the target object. When the artifacts caused by motion are large or affect lesion analysis, the user may perform repeated scans on the target object to improve diagnostic efficiency.

In addition, for medical imaging that cannot intuitively distinguish whether the abnormal results are caused by motion or disease on the generated images, such as spectroscopy and ASL imaging, according to the embodiments of the present disclosure, the user can determine whether the abnormal results on the medical image are caused by motion or disease based on the displayed motion status information, thereby improving the accuracy of diagnosis.

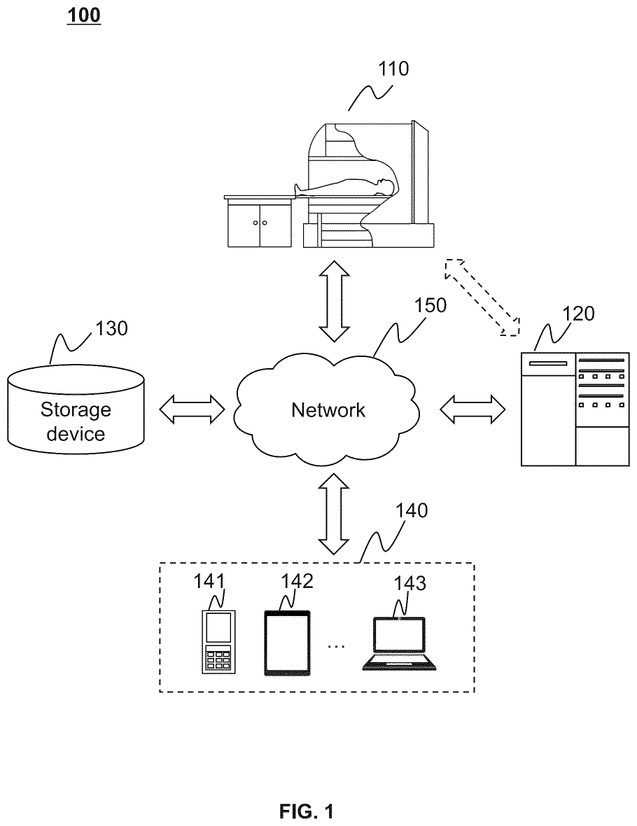

FIG. 1 is a schematic diagram illustrating an exemplary medical imaging system according to some embodiments of the present disclosure. As shown in FIG. 1, the medical imaging system 100 may include a medical imaging device 110, a processing device 120, a storage device 130, a terminal 140, and a network 150. In some embodiments, the medical imaging device 110, the processing device 120, the storage device 130, and/or the terminal 140 may be connected to and/or communicate with each other via a wireless connection, a wired connection, or a combination thereof.

The medical imaging device 110 may be configured to acquire image data relating to at least one part of a target object. The medical imaging device 110 may scan the target object or a portion thereof that is located within its detection region and generate image data relating to the target object or the portion thereof. The image data relating to at least one part of a target object may include one or more images, projection data, or a combination thereof. In some embodiments, the image data may be two-dimensional (2D) image data, three-dimensional (3D) image data, four-dimensional (4D) image data, or the like, or any combination thereof. In some embodiments, the medical imaging device 110 may include a single modality imaging device. For example, the medical imaging device 110 may include a magnetic resonance imaging (MRI) device (also referred to as an MR device, an MR scanner), a computed tomography (CT) device, a digital subtraction angiography (DSA), a positron emission tomography (PET) device, a single-photon emission computed tomography (SPECT) device, an ultrasonography scanner, a digital radiography (DR) scanner, or the like, or any combination thereof. In some embodiments, the medical imaging device 110 may include a multi-modality imaging device. Exemplary multi-modality imaging devices may include a PET-MR device, a PET-CT device, or the like, or a combination thereof. For illustration purposes, the following descriptions are provided with reference to an MRI device, and this is not intended to be limiting.

In some embodiments, the target object includes a human being (e.g., a patient), an animal, or a specific portion, organ, and/or tissue thereof. Merely by way of example, the target object includes head, chest, abdomen, heart, liver, upper limbs, lower limbs, or the like, or any combination thereof. In the present disclosure, the term “object” or “subject” are used interchangeably in the present disclosure.

The processing device 120 may process data and/or information obtained from other device(s) or system component(s) (e.g., the medical imaging device 110, the storage device 130, the terminal 140) and/or perform the medical imaging shown in some embodiments of the present disclosure based on the data, information and/or a processing result to complete one or more of the functions described in some embodiments of the present disclosure. For example, the processing device 120 may obtain motion state information of a region of interest (ROI) of the target object during the scanning process of the target object, wherein the scanning process includes multiple scan phases that are scanned sequentially. During the scanning process, the processing device 120 may display the motion state information corresponding to at least one of multiple scan phases that has been scanned. After the scanning process is completed, he processing device 120 may display the motion state information corresponding to the multiple scan phases and a medical image of the target object obtained in the scanning process.

In some embodiments, the processing device 120 is a computer, a user console, a single server, or a server group. The server group may be centralized or distributed. In some embodiments, the processing device 120 may include a central processing unit (CPU), a digital signal processor (DSP), a system on a chip (SoC), a microcontroller unit (MCU), or the like, or any combination thereof. In some embodiments, the processing device 120 may be local or remote. In some embodiments, the processing device 120 may be implemented on a cloud platform. In some embodiments, the processing device 120 or a portion of the processing device 120 may be integrated into the medical imaging device 110 and/or the terminal 140. In other words, the medical imaging device 110 and/or the terminal 140 may replace the processing device 120 or the portion of the processing device 120 to complete one or more of the functions described in some embodiments of the present disclosure. It should be noted that the processing device 120 in the present disclosure may include one or multiple processors. Thus operations and/or method steps that are performed by one processor may also be jointly or separately performed by the multiple processors.

The storage device 130 may store data, instructions, and/or any other information. In some embodiments, the storage device 130 may store data obtained from the medical imaging device 110, the processing device 120, and/or the terminal 140. In some embodiments, the storage device 130 may store data and/or instructions that the processing device 120 may execute or use to perform exemplary methods described in the present disclosure. In some embodiments, the storage device 130 may include a mass storage device, a removable storage device, a volatile read-and-write memory, a read-only memory (ROM), or the like, or a combination thereof. In some embodiments, the storage device 130 may be implemented on a cloud platform. In some embodiments, the storage device 130 may be part of the medical imaging device 110, the processing device 120, or the terminal 140.

The terminal 140 may be configured to enable user interactions between a user and the medical imaging system 100. In some embodiments, the terminal 140 may be connected to and/or communicate with the medical imaging device 110, the processing device 120, and/or the storage device 130. In some embodiments, the terminal 140 may include a mobile device 141, a tablet computer 142, a laptop computer 143, or the like, or a combination thereof. In some embodiments, the terminal 140 may be part of the processing device 120 and/or the medical imaging device 110.

The network 150 may include any suitable network that can facilitate the exchange of information and/or data for the medical imaging system 100. In some embodiments, one or more components of the medical imaging system 100 (e.g., the medical imaging device 110, the processing device 120, the storage device 130, the terminal 140, etc.) may communicate information and/or data with one or more other components of the medical imaging system 100 via the network 150.

It should be noted that the above description is intended to be illustrative, and not to limit the scope of the present disclosure. Many alternatives, modifications, and variations will be apparent to those skilled in the art. The features, structures, methods, and characteristics of the exemplary embodiments described herein may be combined in various ways to obtain additional and/or alternative exemplary embodiments. In some embodiments, the medical imaging system 100 may include one or more additional components and/or one or more components described above may be omitted. Additionally or alternatively, two or more components of the medical imaging system 100 may be integrated into a single component. For example, the processing device 120 may be integrated into the medical imaging device 110. As another example, a component of the medical imaging system 100 may be replaced by another component that can implement the functions of the component. However, those variations and modifications do not depart from the scope of the present disclosure.

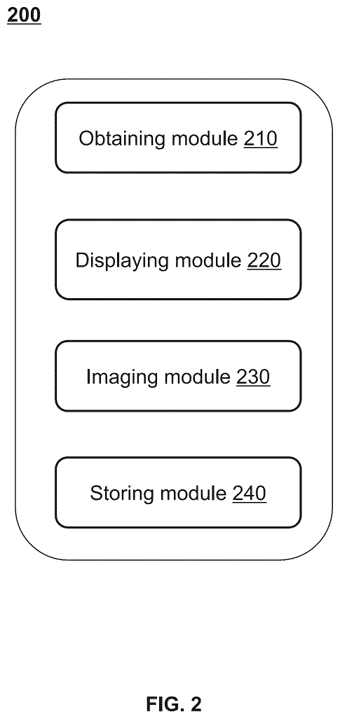

FIG. 2 is a block diagram illustrating an exemplary processing device according to some embodiments of the present disclosure. As shown in FIG. 2, the processing device 120 may include an obtaining module 210, a displaying module 220, and an imaging module 230, and a storing module 240.

The obtaining module 210 may be configured to obtain motion state information of a region of interest (ROI) of a target object during a scanning process of the target object. The scanning process includes multiple scan phases that are scanned sequentially. More descriptions of the obtaining of the motion state information may be found elsewhere in the present disclosure (e.g., operation 310 and the descriptions thereof).

The displaying module 220 may be configured to display, during the scanning process, the motion state information corresponding to at least one of multiple scan phases that has been scanned. The displaying module 220 may be configured to display, after the scanning process is completed, the motion state information corresponding to the multiple scan phases and the medical image of the target object obtained in the scanning process. More descriptions of the displaying of the motion state information may be found elsewhere in the present disclosure (e.g., operations 320-330 and the descriptions thereof).

The imaging module 230 may be configured to perform the scanning process to obtain the medical image of the target object.

The storing module 240 may be configured to store, based on a preset storage rule, the medical image of the target object and the motion state information corresponding to the multiple scan phases. More descriptions of the storing of the medical image of the target object and the motion state information may be found elsewhere in the present disclosure (e.g., operation 860 and the descriptions thereof).

It should be noted that the above description is merely provided for the purposes of illustration, and is not intended to limit the scope of the present disclosure. For persons having ordinary skills in the art, multiple variations and modifications may be made under the teachings of the present disclosure. However, those variations and modifications do not depart from the scope of the present disclosure. In some embodiments, the processing device 120 may include one or more additional modules, such as a storage module (not shown) for storing data.

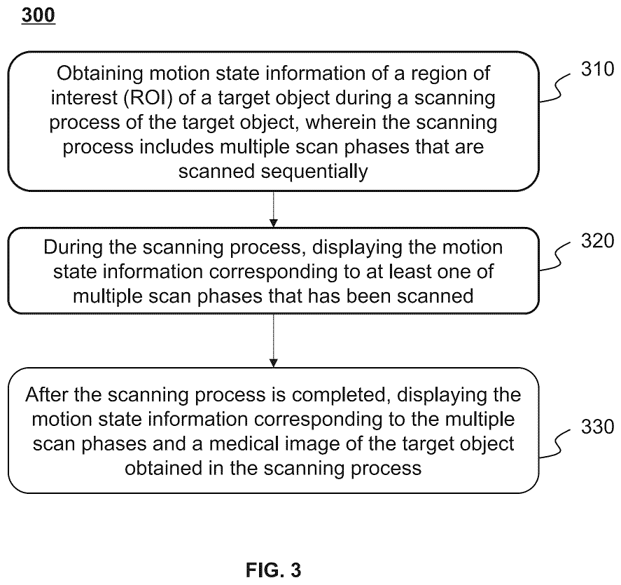

FIG. 3 is a flowchart illustrating an exemplary process for medical imaging according to some embodiments of the present disclosure. In some embodiments, process 300 may be executed by the medical imaging system 100. For example, process 300 may be implemented as a set of instructions (e.g., an application) stored in a storage device (e.g., the storage device 130), and the processing device 120 (e.g., one or more modules illustrated in FIG. 2) may execute the set of instructions and may accordingly be directed to perform the process 300.

In 310, the processing device 120 (e.g., the obtaining module 210) may obtain motion state information of a region of interest (ROI) of a target object during a scanning process of the target object.

The ROI refers to a target imaging part (e.g., the head, the abdomen, etc.) of the target object or a part of the target object whose movement will affect the imaging of the target imaging part. The motion state information refers to information that can reflect a position change of the target imaging part of the target object in each scan phase. The motion state information represents a motion trend of the ROI of the target object in each scan phase. Merely by way of example, the motion state information includes at least one of a movement probability, a movement level, a motion offset, a motion direction, a motion curve, or the like, or a combination thereof. The movement probability refers to a probability of ROI moving. In some embodiments, the movement probability is expressed as numbers (0, 0.1, 0.5, 0.8, 1, etc.), percentages (e.g., 0%, 10%, 50%, 80%, 100%, etc.). The movement level is a level obtained by dividing the probability of the ROI moving. Merely by way of example, the movement level may include high, medium, low, etc. The motion offset refers to the amount by which the ROI deviates from its original position. In some embodiments, the motion offset is expressed as numbers (0, 1 mm, 5 mm, 8 mm, 1 mm, etc.). The motion direction refers to a direction in which the ROI moves relative to its original position. The motion curve refers to a graph that can reflect the motion state of the target object during scanning process, for example, a curve with the abscissa of time and the ordinate of a value of the movement probability or the motion offset.

The scanning process refers to a process of medical imaging of the target object. Merely by way of example, the scanning process includes a magnetic resonance imaging (MRI) process, a computed tomography (CT) imaging process, a digital subtraction angiography (DSA) imaging process, a positron emission tomography (PET) imaging process, a single-photon emission computed tomography (SPECT) imaging process, an ultrasonography imaging process, a digital radiography (DR) imaging process, or the like, or any combination thereof. For illustration purposes, the following descriptions are provided with reference to an MRI process, and this is not intended to be limiting.

The scanning process may include multiple scan phases that are scanned sequentially. In some embodiments, the scanning process is divided arbitrarily or evenly into the multiple scan phases that are scanned sequentially. In other words, scan times of the multiple scan phases may be equal or different. For example, for MRI process, an MRI sequence (also referred to as a scanning sequence) is used for imaging by a medical imaging device (e.g., the medical imaging device 110). The MRI sequence may include a plurality of imaging sub-sequences that are performed by the medical imaging device sequentially. Each imaging sub-sequence may correspond to a repetition time (TR). The TR may be a scan phase.

In some embodiments, the processing device 120 may obtain scanning information of the target object from the scanning process (e.g., the imaging sequence). The scanning information may include image data relating to the target object and position information of the target object in each scan phase that has been scanned. In some embodiments, the processing device 120 may obtain the position information of the target object in each scan phase that has been scanned by continuously transmitting echo signals or laser ranging signals to the target object. For the each scan phase that has been scanned, the processing device 120 may determine the motion state information of the ROI corresponding to the scan phase by comparing the position information of the target object corresponding to the scan phase and the initial position information of the target object. For example, the processing device 120 compares the position information of the target object corresponding to the scan phase and the initial position information of the target object using a comparison manner and determines the motion state information of the ROI corresponding to the scan phase based on the comparing result. Merely by way of example, the comparison manner includes a difference manner, a percentage manner, a mean manner, a median manner, a standard deviation manner, or the like, or any combination thereof, and accordingly, the comparing result includes a difference, a percentage value, a mean, a median, or a standard deviation. In some embodiments, the processing device 120 may designate the comparing result corresponding to the ROI as the motion state information of the ROI.

In some embodiments, the processing device 120 may obtain the motion state information by scanning the target object using a motion detection device inserted into the multiple scan phases in the scanning process. The motion detection device may be a device (or operation) used for motion detection by the medical imaging device. For example, for the MRI process, the motion detection device may be a magnetic resonance scan sequence (or operation) used for motion detection by the MRI device. In such cases, the motion detection device may be referred to as an auxiliary sequence (also referred to as a motion detection sequence), and the multiple scan phases may be TRs of the MRI sequence. The processing device 120 may obtain auxiliary MR data of the target object by scanning the target object using the auxiliary sequence inserted in at least two imaging sub-sequences of the MRI sequence, and determine the motion state information based on the auxiliary MR data. The auxiliary sequence may include a plurality of auxiliary sub-sequences inserted at different positions in the at least two imaging sub-sequences. In some embodiments, the processing device 120 may scan the target object using the imaging sub-sequence(s) and/or the auxiliary sequence(s) through the medical imaging device. The imaging sub-sequence(s) may be used to obtain magnetic resonance imaging data of the target object, and the auxiliary sequence may be used to obtain the auxiliary magnetic resonance data of the target object. The magnetic resonance imaging data refers to data corresponding to magnetic resonance signal(s) used to obtain a medical image. The auxiliary magnetic resonance data refers to data corresponding to magnetic resonance signal(s) used to determine the motion state information.

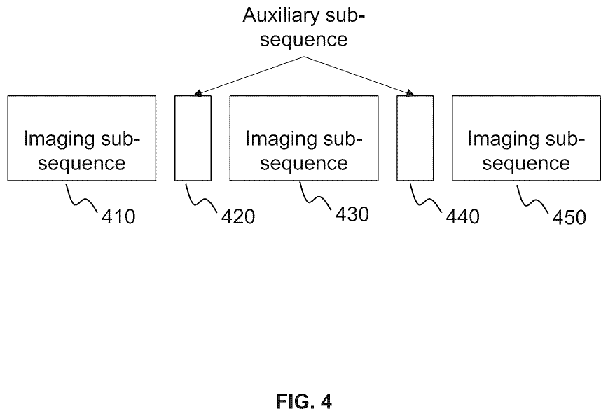

In some embodiments, an auxiliary sub-sequence may be inserted in a TR and adjacent to the imaging sub-sequence in the TR. For example, at least one auxiliary sub-sequence may be inserted before or after the imaging sub-sequence in the TR. Therefore, at least one auxiliary sub-sequence may be inserted between two adjacent imaging sub-sequences, and the two adjacent imaging sub-sequences may belong to two adjacent TRs, respectively. FIG. 4 is a schematic diagram illustrating imaging sub-sequences and auxiliary sub-sequences according to some embodiments of the present disclosure. As shown in FIG. 4, the auxiliary sub-sequence 420 may be inserted between the imaging sub-sequences 410 and 430, and the auxiliary sub-sequence 440 may be inserted between the imaging sub-sequences 430 and 450. Each of the imaging sub-sequences 410, 430, and 450 may correspond to a TR.

Further, the processing device 120 may determine the data of the ROI from the obtained auxiliary magnetic resonance data and determine the motion state information of the ROI of the target object according to the data of the RO. More descriptions regarding the motion detection of the target object may be found in, for example, U.S. patent application Ser. No. 18/313,334, entitled “METHODS AND SYSTEMS FOR MOTION DETECTION IN MAGNETIC RESONANCE IMAGING,” filed on May 6, 2023, the contents of which are hereby incorporated by reference.

In 320, during the scanning process, the processing device 120 (e.g., the displaying module 220) may display the motion state information corresponding to at least one of multiple scan phases that has been scanned.

During the scanning process, the processing device 120 may display the motion state information in real time. For example, whenever one scan phase is completed, the processing device 120 may update the display in real time based on the latest obtained motion status information.

The motion state information of the target object is displayed in real time during the scanning process, which can help to better control the scanning process without affecting the imaging effect of the medical imaging when the motion amplitude of the target object is relatively large. For example, a user (such as a doctor) may obtain the motion status information of the target object in real time based on the first visual chart, and when the motion amplitude of the target object is relatively large, the user may terminate the scanning process in advance or promptly remind the target object to reduce movement, which can avoid wasting scanning time, improve efficiency, thereby avoiding the impact (e.g., artifacts) of the target object's motion on the final generated medical image, resulting in the need to rescan the target object, and accordingly reducing the increase in radiation exposure to the target object caused by secondary scanning due to serious artifacts caused by motion after image generation.

In some embodiments, the processing device 120 may form, based on an execution order of the at least one of multiple scan phases, a first visual chart of the motion state information corresponding to the at least one of multiple scan phases that has been scanned. In the first visual chart, the abscissa corresponds to the at least one scan phase that has been scanned, and the ordinate corresponds to the corresponding motion status information (e.g., the movement probability, the motion offset). For example, the abscissa may be the scan time or scan progress of the at least one scan phase that has been scanned. As another example, the abscissa may be shot order or motion detection order of the of the at least one scan phase that has been scanned. As a further example, for the MRI process, the abscissa may be each TR that has been scanned. The further the ordinate deviates from the origin, for example, the ordinate is higher than the origin or the ordinate is lower than the origin, the greater the corresponding motion status information (e.g., the movement probability, the motion offset).

In some embodiments, the first visual chart may add a title associated with the scanning process based on actual needs. For example, the title of the first visual chart may be a number and/or name of the MRI sequence, or any other information that can help determine the current MRI sequence. The title of the first visual chart may be located anywhere on the first visual chart or on a display interface of the processing device 120 that is used to display the first visual chart.

In some embodiments, a size of the first visual chart for different scanning processes may be set differently or the same, or the size of the first visual chart may be adaptively and dynamically adjusted according to different scanning processes. In some embodiments, if the number of the at least one scan phase that has been scanned is large, the first chart may not be fully displayed in the display interface. In such cases, a sliding bar may appear on the display interface, and the sliding bar may be slid to fully display the first chart. If there is a sliding bar, the display interface may support an automatic sliding display according to the order of the at least one scan phase that has been scanned.

In some embodiments, the first chart may be stored in a form of Dicom (Digital Imaging and Communications in Medicine) to facilitate subsequent loading and calling of the first chart. In some embodiments, the first chart may be called up in any way. For example, the first chart may be called up in the form of a right-click menu. In some embodiments, the first chart may be placed at any suitable location on the display interface or displayed in a pop-up window. The display and hiding of the first chart may be freely controlled through an appropriate switch or operation.

In some embodiments, the first visual chart may be a 2D or 3D chart. In some embodiments, the first visual chart includes a scatter chart, a line chart, a bar chart, or the like, or any combination thereof.

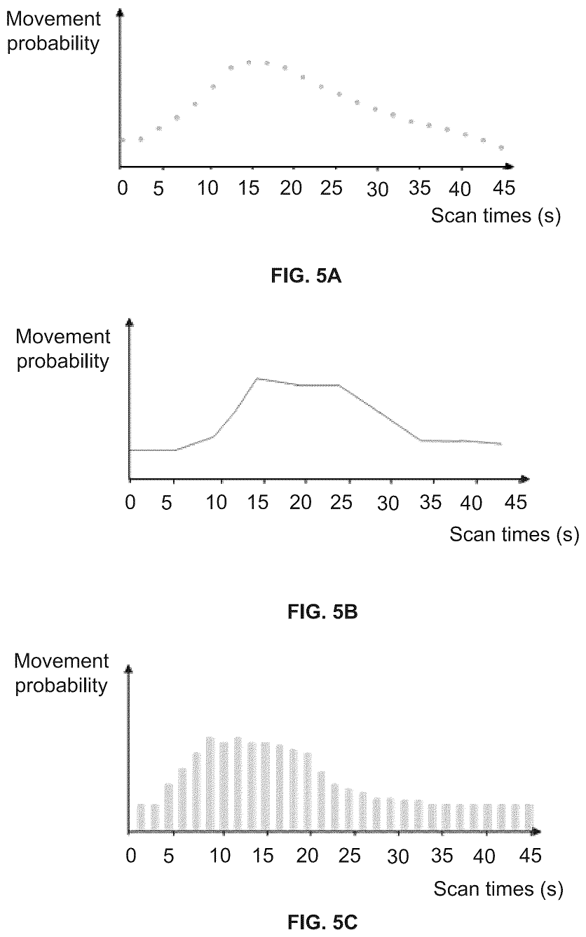

FIGS. 5A-5C are schematic diagrams each of which illustrates an exemplary first visual chart according to some embodiments of the present disclosure. As shown in FIG. 5A, the first visual chart is a scatter chart, in which the abscissa corresponds to the scan times of the scan phases that have been scanned, and the ordinate corresponds to the corresponding movement probability. In FIG. 5A, the higher the ordinate of a point corresponding to each scan phase, the greater the probability that the target object will move in that scan phase. As shown in FIG. 5B, the first visual chart is a line chart, in which the abscissa corresponds to the scan times of the scan phases that have been scanned, and the ordinate corresponds to the corresponding movement probability. In FIG. 5B, the higher the ordinate of a line corresponding to each scan phase, the greater the probability that the target object will move in that scan phase. As shown in FIG. 5C, the first visual chart is a bar chart, in which the abscissa corresponds to the scan times of the scan phases that have been scanned, and the ordinate corresponds to the corresponding movement probability. In FIG. 5C, the higher the ordinate of a column corresponding to each scan phase, the greater the probability that the target object will move in that scan phase.

Further, the processing device 120 may display the first visual chart. Specifically, during the scanning process, the processing device 120 may display the first visual chart in real time. In some embodiments, whenever one scan phase is completed, the processing device 120 may update the first visual chart in real time based on the latest obtained motion status information and display the latest updated first visual chart.

According to the embodiments of the present disclosure, during the scanning process, the processing device 120 may display, in chart form, the motion state information corresponding to the scan phases that have been scanned in real time, which is more intuitive and eye-catching, allowing the user to more conveniently and timely obtain the motion status of the target object during the scanning process.

In some embodiments, the processing device 120 may obtain a preset threshold and highlight elements of the first visual chart corresponding to the motion state information based on the preset threshold.

The preset threshold may be related to a scan site of the ROI of the target object. Different scan sites correspond to different preset thresholds. The movement of the scan site includes active movement and passive movement. The active movement refers to a movement that the target object can control, such as limb movement. The passive movement refers to movement that is not controlled by the target object, such as breathing, swallowing, gastrointestinal motility, etc. The passive movement is not controlled by the target object, so the preset threshold corresponding to the scan site where passive movement occurs may be higher than he preset threshold corresponding to the scan site where active movement occurs.

If the medical image of a specific scan site is more susceptible to the influence of motion, the preset threshold corresponding to the scan site is lower. If the medical image of a specific scan site is less susceptible to the influence of motion, the preset threshold corresponding to the scan site is higher. For example, if imaging of a specific scan site requires higher image contrast and/or resolution, the preset threshold corresponding to that scan site will be lower. Therefore, the preset threshold also is related to the contrast and/or resolution required for the final generated medical image. Different contrast and/or resolution requirements correspond to different preset thresholds. Medical images obtained by different imaging sequences have different contrasts and/or resolutions, therefore the preset threshold also is related to the imaging sequence. Different imaging sequences correspond to different preset thresholds.

In some embodiments, the processing device 120 may determine a first preset threshold based on the scan site, a second preset threshold based on the contrast requirement, a third preset threshold based on the resolution requirement, and a forth preset threshold based on the imaging sequence. Then, the processing device 120 may determine the preset threshold based on the first preset threshold, the second preset threshold, the third preset threshold, and the fourth preset threshold. For example, the processing device 120 may determine a median, a mean, or a weighted mean of the first preset threshold, the second preset threshold, the third preset threshold, and the fourth preset threshold as the preset threshold. The processing device 120 may determine weights of the first preset threshold, the second preset threshold, the third preset threshold, and the fourth preset threshold based on the historical imaging data. For example, when each of the first preset threshold, the second preset threshold, the third preset threshold, and the fourth preset threshold is used as the preset threshold, in the first chart, the processing device 120 may determine a first ratio of the number of scan phases where the motion status information exceeds the preset threshold to the total number of scan phases. Further, the processing device 120 may determine a second ratio of the number of scan phases corresponding to the abnormal image areas in the final generated medical image to the total number of scan phases. Then, the processing device 120 may determine a difference between the first ratio and the second ratio. The larger the difference, the smaller the weight of the corresponding threshold.

In some embodiments, the processing device 112 may retrieve the preset threshold from a preset table based on the scan site, the contrast requirement, the resolution requirement, and the imaging sequence. The preset table may include a corresponding relationship between preset thresholds and groups each of which includes a scan site, a contrast requirement, a resolution requirement, and an imaging sequence.

In some embodiments, the processing device 120 may determine the preset threshold based on the scan site, the contrast requirement, the resolution requirement, and the imaging sequence using a trained machine learning model (e.g., a neural network model). For example, the processing device 120 may input the scan site, the contrast requirement, the resolution requirement, and the imaging sequence into the trained machine learning model, and designate an output of the the trained machine learning model as the preset threshold. The trained machine learning model may be obtained based on a plurality of groups of training data. Each group of training data may include a sample scan site, a sample contrast requirement, a sample resolution requirement, and a sample imaging sequence that are obtained from historical imaging data. During the training, the corresponding preset threshold retrieved from the preset table may be used as a label.

In some embodiments, different scan phases correspond to different preset thresholds. For example, in the early stage of the scanning process, the probability of the target object moving is low, so the preset threshold corresponding to the scan phase in the early stage may be relatively high. In the later stage of the scanning process, the probability of the target object moving is low, so the preset threshold corresponding to the scan phase in the later stage may be relatively low.

According to the embodiments of the present disclosure, the setting of the preset threshold is associated with the scan site, the contrast requirement, the resolution requirement, and the imaging sequence, making the preset threshold more in line with actual medical imaging requirements, avoiding false reminders and imaging interruptions caused by setting too small a preset threshold, or avoiding poor imaging effect of the generated medical image caused by setting too large a preset threshold.

Further, the processing device 120 may highlight the elements of the first visual chart corresponding to the motion state information based on the preset threshold. For example, the processing device 120 may highlight the elements of the first visual chart corresponding to the motion state information closing to and/or exceeding the preset threshold to prompt the user that the target object may have moved. Merely by way of example, the elements of the first visual chart may include points, lines, columns, etc. representing motion status information in the first visual chart. In some embodiments, the manner of highlighting the elements of the first visual chart includes changing, according to a distance between the motion status information and the preset threshold, a color, a thickness, an outline, a size, or a style of the elements to more intuitively display the motion status of the target object to the user. For example, when the first visual chart is the scatter chart, the processing device 120 may highlight the elements by changing the color, outline, shape, size, fill style, outline, etc. of the points. As another example, when the first visual chart is the line chart, the processing device 120 may highlight the elements by changing the color, thickness, outline, dashed and solid lines, etc. of the lines. As a further example, when the first visual chart is the bar chart, the processing device 120 may highlight the elements by changing the color, outline, fill style, size, etc. of the column. In some embodiments, the processing device 120 may change the color by using gradients or solid colors.

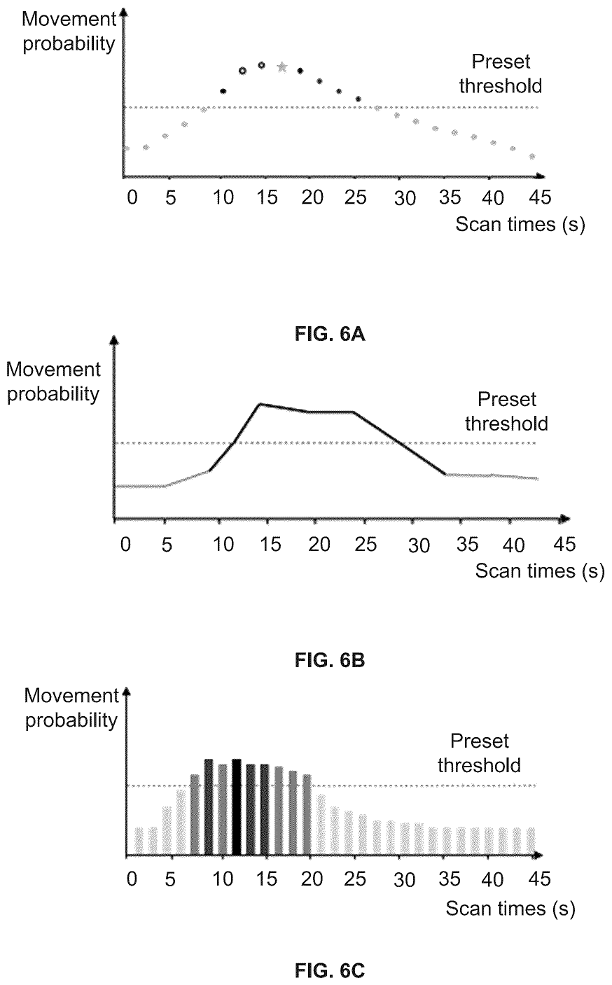

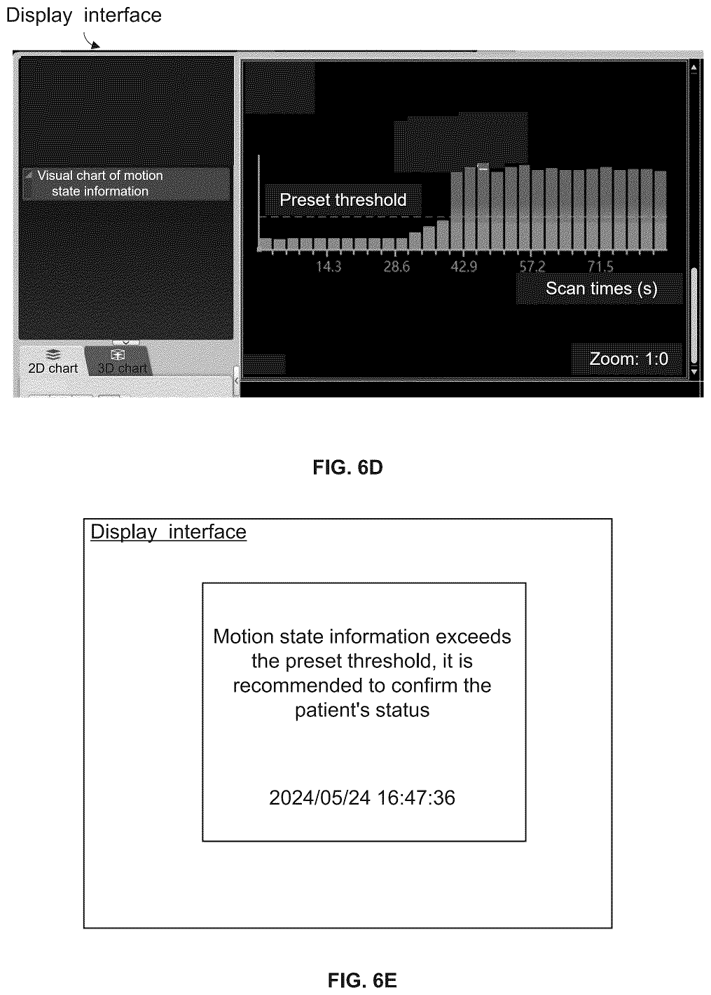

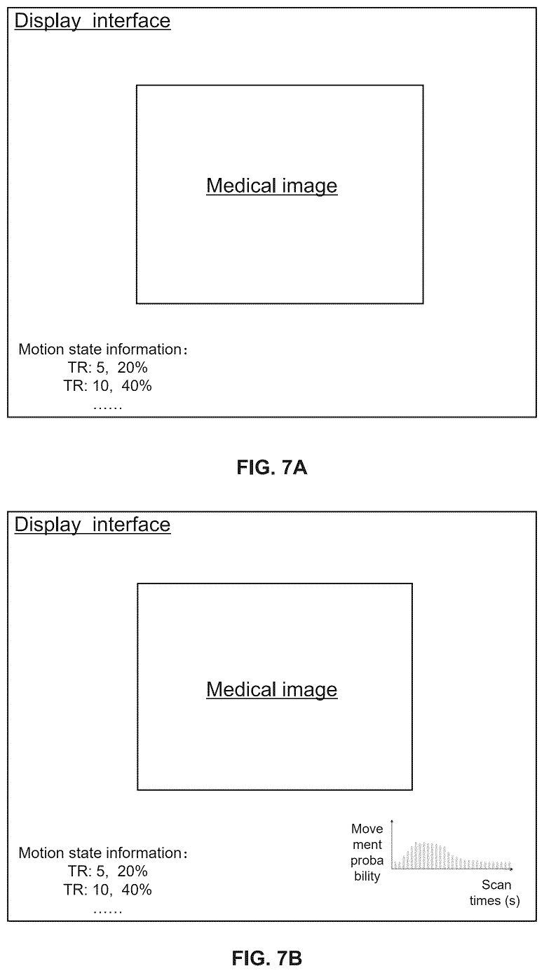

FIGS. 6A-6C are schematic diagrams each of which illustrates an exemplary first visual chart with highlighting elements according to some embodiments of the present disclosure. As shown in FIG. 6A, when the first visual chart is the scatter chart, points corresponding to motion status information larger than the preset threshold are displayed using at least one of different shapes, colors, fill styles, and outlines from other points to remind the user of possible movements of the target object in the corresponding scan phases. As shown in FIG. 6B, when the first visual chart is the line chart, line segments corresponding to motion status information close to and larger than the preset threshold are displayed with thicker lines than other line segments to remind the user of possible movements of the target object in the corresponding scan phases. As shown in FIG. 6C, when the first visual chart is the bar chart, the column corresponding to the motion status information higher than the preset threshold is displayed using at least one of different sizes, color, fill styles, and outlines from other columns to remind the user of possible movements of the target object in the corresponding scan phases.

FIG. 6D is a schematic diagrams illustrating an exemplary first visual chart according to some embodiments of the present disclosure. As shown in FIG. 6E, the first visual chart is display on the display interface of the processing device 120, and the first visual chart is a 2D or 3D chart. The first visual chart may be zoomed to a certain ratio on the display interface. It should be noted that not all medical imaging data corresponding to the motion status information exceeding the preset threshold cannot be used to generate the medical image. For example, as shown in FIG. 6D, after movement occurs at 42.9 s, the movement of the target object in the subsequent scan time (42.9 s-71.5 s) is in a relatively stable state, which has less impact on the quality of the medical image. Therefore, the medical imaging data obtained during subsequent scanning times may still be used to generate the medical image, but the medical imaging data in the junction area (i.e. 42.9 s) needs to be discarded.

In some embodiments, the processing device 120 may send warning information to the user and/or the target object based on the preset threshold. For example, when the motion state information closes to and/or exceeds the preset threshold, the processing device 120 may send the warning information to the user and/or the target object to remind the target object to remain static. After receiving the reminder information, the user may choose to continue the scanning process or remind the target object to remain static. Merely by way of example, the warning information includes but is not limited to light signal prompts, voice prompts, image prompts, or page pop-ups. FIG. 6E is a schematic diagrams illustrating exemplary warning information according to some embodiments of the present disclosure. As shown in FIG. 6E, the processing device 120 may send the warning information in the form of a pop-up window on the display interface of the processing device 120, and the warning information includes that the motion state information exceeds the preset threshold, it is recommended to confirm the patient's status. Further, the warning information includes the time when the warning information was issued.

According to the embodiments of the present disclosure, during the scanning process, when the motion state information closes to and/or exceeds the preset threshold, the processing device 120 may remind the user and/or the target object in real time in a variety of ways, for example, highlighting the elements of the first visual chart, sending warning information. As a result, the target object can stop moving in time to reduce the movement during the subsequent scanning process and improve the imaging effect.

In 330, after the scanning process is completed, the processing device 120 (e.g., the displaying module 220) may display the motion state information corresponding to the multiple scan phases and the medical image of the target object obtained in the scanning process.

In some embodiments, after the scanning process is completed, the processing device 120 may generate the medical image of the target object based on the image data obtained in the scanning process. Further, the processing device 120 may display the motion status information corresponding to all scan phases and the medical image together.

After the scanning process is completed, the motion state information and the obtained medical image of the target object may be displayed together, which makes it easier for the user to analyze the medical image. For example, When analyzing the medical image, the user can intuitively understand the correlation between the motion status information and the medical image, he/she can refer to the motion status information to determine whether the abnormalities (e.g., artifacts) in the corresponding medical image are caused by the movement of the target object. When abnormalities caused by motion are small or do not affect lesion analysis, the user may make a diagnosis without repeating scans of the target object. When the abnormalities caused by motion are large or affect lesion analysis, the user may perform repeated scans on the target object to improve diagnostic efficiency. In addition, for medical imaging that cannot intuitively distinguish whether the abnormal results are caused by motion or disease on the generated images, such as spectroscopy and ASL imaging, according to the embodiments of the present disclosure, the user can determine whether the abnormal results on the medical image are caused by motion or disease based on the displayed motion status information, thereby improving the accuracy of diagnosis.

In some embodiments, after the scanning process is completed, the processing device 120 may display, based on a preset display rule in a same display interface, the motion state information corresponding to the multiple scan phases and the medical image of the target object.

By displaying the motion status information and the medical image together on the same display interface, the user can simultaneously compare and view the motion status information and the medical image, thereby improving the efficiency of the user in analyzing the medical image and making diagnoses.

In some embodiments, the preset display rule may include displaying the motion state information corresponding to the multiple scan phases in a displaying region on the display interface. The displaying region may include a region, in the display interface, not occupied by the medical image, at least one of four corners of the medical image, or a pop-up window of the display interface. By displaying the motion state information in the displaying region on the display interface, the motion state information is displayed in a simple way, keeping the medical image clean and clear while improving the information density of the medical image on the display interface.

In some embodiments, the preset display rule may include displaying the motion state information exceeding the preset threshold in the displaying region. The motion status information exceeding the preset threshold has a high probability of causing abnormalities in the medical image. By displaying the motion state information exceeding the preset threshold, the user can focus on it, thereby improving the efficiency and accuracy of the user in analyzing the medical image and making diagnoses.

In some embodiments, the preset display rule may include forming, based on an execution order of the multiple scan phases, a second visual chart of the motion state information corresponding to the multiple scan phases, and displaying the second visual chart in the displaying region. After the scanning process is completed, all scan phases have been scanned, the processing device 120 may form the second visual chart of the motion state information corresponding to all scan phases. The second visual chart is similar to the first visual chart. The difference between the second visual chart and the first visual chart is that the first visual chart is formed during the scanning process and corresponds to the scanning interval of the current completed scan, and the second visual chart is formed after the scanning process is completed and corresponds to all scan phases of the completed scans. More descriptions of the first visual chart may be found elsewhere in the present disclosure (e.g., operation 320 and the descriptions thereof). In the displaying region, the motion state information corresponding to all scan phases of the completed scans is displayed in chart form, which is more intuitive and eye-catching, allowing the user to more conveniently analyze the medical image and make diagnoses.

In some embodiments, the preset display rule may include displaying the second visual chart in the displaying region, and highlighting elements of the second visual chart corresponding to the motion state information exceeding the preset threshold. The operation of highlighting elements of the second visual chart is similar to the operation of highlighting elements of the first visual chart, more descriptions may be found elsewhere in the present disclosure (e.g., operation 320 and the descriptions thereof). By highlighting elements of the second visual chart, when analyzing the medical image, the user can be reminded of the motion state information close to and/or exceeds the preset threshold, thereby improving the efficiency and accuracy of the user in analyzing the medical image and making diagnoses.

In some embodiments, the preset display rule may include marking an image region corresponding to the motion state information exceeding the preset threshold in the medical image. By marking the image region corresponding to the motion state information exceeding the preset threshold in the medical image, when analyzing the medical image, the user can be reminded of the correlation between an abnormal region in the medical image and the movement of the target object, thereby improving the efficiency and accuracy of the user in analyzing the medical image and making diagnose.

In some embodiments, the preset display rule may include marking the motion state information into the corresponding image region of the medical image. By marking the motion state information into the corresponding image region of the medical image, when analyzing the medical image, the user can be reminded of the correlation between each region of the medical image and the movement of the target object, thereby improving the efficiency and accuracy of the user in analyzing the medical image and making diagnose.

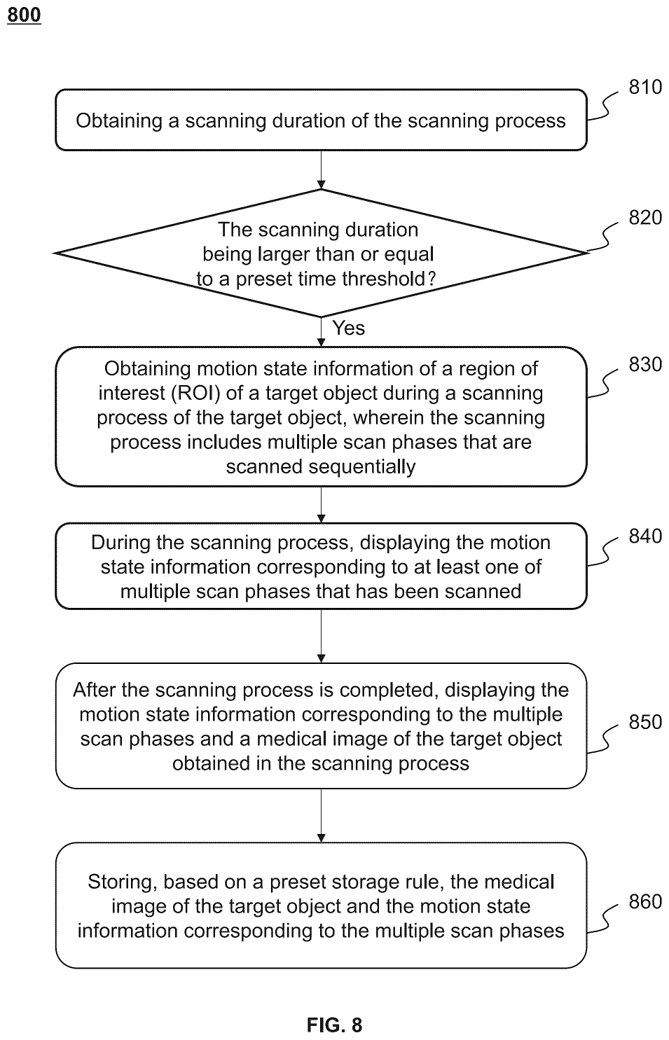

FIGS. 7A-7B are schematic diagrams each of which illustrates an exemplary display interface according to some embodiments of the present disclosure. As shown in FIG. 7A, the medical image is displayed in a middle region of the display interface, and the motion state information is displayed in a lower left corner of the display interface. In the displayed motion state information, 20% and 40% are movement probabilities, 5 and 10 are the corresponding TRs. As shown in FIG. 7B, the medical image is displayed in the middle region of the display interface, the motion state information is displayed in the lower left corner of the display interface, and the second visual chart is displayed in the lower right corner of the display interface.

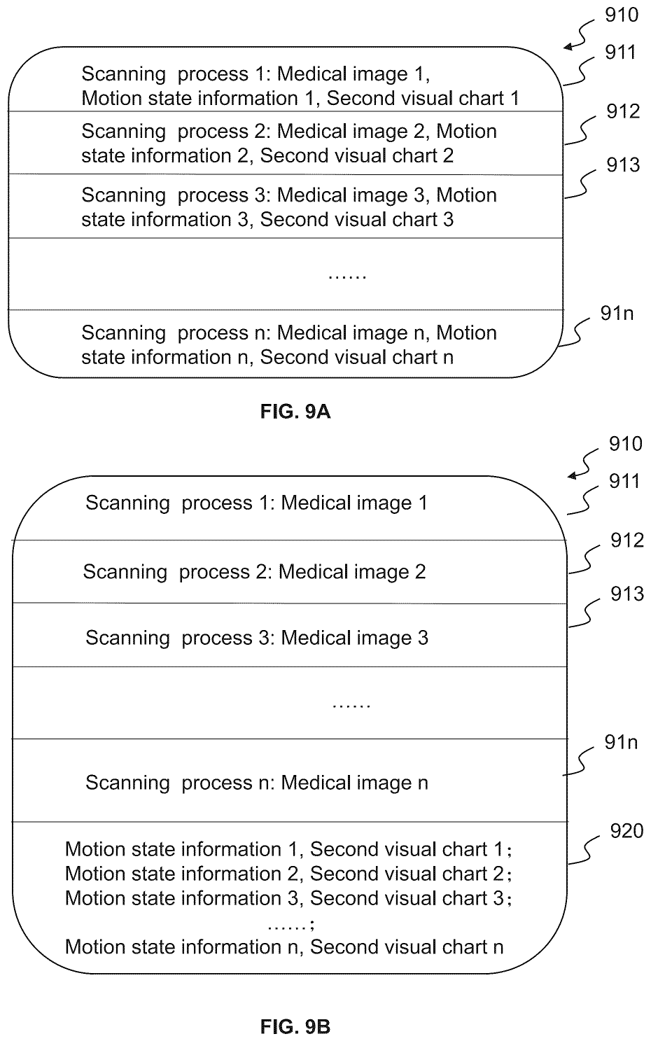

In some embodiments, the processing device 120 may store, based on a preset storage rule, the medical image of the target object and the motion state information corresponding to the multiple scan phases. More descriptions of the storing of the medical image and the motion state information may be found elsewhere in the present disclosure (e.g., FIG. 8 and the descriptions thereof).

In some embodiments, before performing operation 310, the processing device 120 may obtain a scanning duration of the scanning process. In response to the scanning duration being larger than or equal to a preset time threshold, the processing device 120 may perform operation 310 to obtain the motion state information of the ROI of the target object during the scanning process. In response to the scanning duration being less than the preset time threshold, the processing device 120 may perform the scanning process directly to obtain the medical image of the target object. More descriptions of operations before operation 310 may be found elsewhere in the present disclosure (e.g., FIG. 8 and the descriptions thereof).

In some embodiments, after the scanning process is completed, the processing device 120 may only display the medical image of the target object obtained in the scanning process, and the motion state information corresponding to the multiple scan phases may be called up in any way. For example, the motion state information may be called up in the form of a right-click menu.

In some embodiments, after the scanning process is completed, the processing device 120 may determine and display movement level of the target object on the display interface. Merely by way of example, the movement level includes high, medium, low, etc.

In some embodiments, the processing device 120 may determine a first ratio of the number of scan phases where the motion status information exceeds the preset threshold to the total number of scan phases. When the first ratio is larger than or equal to a first preset ratio threshold, the processing device 120 may determine the movement level of the target object is high. When the first ratio is less than the first preset ratio threshold and larger than a second preset ratio threshold, the processing device 120 may determine the movement level of the target object is medium. When the first ratio is less than or equal to the second preset ratio threshold, the processing device 120 may determine the movement level of the target object is low. The preset ratio threshold may be determined based on experience, for example, the first preset ratio threshold is 80%, and the second preset ratio threshold is 20%.

In some embodiments, the processing device 120 may determine the movement level based on the medical image, the motion status information, and the second visual chart using a trained machine learning model (e.g., a U-net model, a 3D U-net model). For example, the trained machine learning model may include an artifact determination layer and a motion determination layer. The processing device 120 may input the medical image into the artifact determination layer and the artifact determination layer may output an artifact level of the medical image. Further, the processing device 120 may input the artifact level of the medical image, the medical image, the motion status information, and the second visual chart into the motion determination layer, and the motion determination layer may output the movement level of the target object.

In the training of the artifact determination layer, each group of training data may include a sample medical image, and the label of training may be manually labeled. In the training of the motion determination layer, experimental data may be selected as sample data. Each two groups in the sample data have a corresponding relationship. Specifically, the same sample object is subjected to medical imaging successively using the same imaging parameters to obtain two medical images. Since there are differences in the motion of the sample object during the two scanning processes, the two medical images have different artifacts, and therefore the artifact levels output by the artifact determination layer based on the two medical images are also different. However, the disease of the sample object itself has not changed much, so it can be considered that the difference in the two medical images is entirely due to the different degree of movement of the sample object. These two medical images are two sets of sample data that have a corresponding relationship. When determining the label, the label of a group of sample data with a high artifact level is determined to be 1. Based on the difference in the artifact levels between the two groups of sample data, the label of a group of sample data with a low artifact level is determined. The greater the difference, the closer the label is to 0.

In some embodiments, after the scanning process is completed, the processing device 120 may determine and display the first ratio of the number of scan phases where the motion status information exceeds the preset threshold to the total number of scan phases. In some embodiments, after the scanning process is completed, the processing device 120 may determine and display movement duration, mean and variance of movement probability, movement score, etc.

In some embodiments, the processing device 120 may perform a personalized display based on the user's operating habits. For example, the processing device 120 automatically determines a display method based on the display methods selected by the user in history. For example, the processing device 120 directly uses the last display method used by the user. As another example, the processing device 120 uses the display method most commonly used by the user.

In some embodiments, the processing device 120 may adjust the display method based on the historical movement of the target object. For example, if the target object has a relatively high degree of movement during the historical scanning processes, the processing device 120 may display the second visual chart of the current medical imaging in a more conspicuous region of the display interface, and the size of the second visual chart is larger. As another example, if the target object has a relatively low degree of motion during the historical scanning processes, the processing device 120 may set the second visual chart of the current medical imaging to a hidden state. When the motion status information is detected to exceed the preset threshold, the second visual chart will pop up in the form of a pop-up window.

In some embodiments, operation 320 and operation 330 both are performed. In some embodiments, one of operation 320 and operation 330 is canceled, that is, only one of operation 320 and operation 330 is executed.

In some embodiments, the processing device 120 may obtain a scanning sequence for scanning a target object. The scanning sequence including multiple scan phases. For at least one of the multiple scan phases, the processing device 120 may obtain motion monitoring signals of the target object by applying a motion detection sequence before the scan phase. Then, the processing device 120 may generate motion state information of the target object based on the motion monitoring signals. The motion state information includes a movement probability or a movement level representing a position change of the target object in the scan phase. Further, the processing device 120 may control a display interface of the medical imaging device to display the movement probability or the movement level. In some embodiments, the display interface sequentially displays movement probabilities or movement levels corresponding to the multiple scan phases in a form of a scatter plot, a line chart, or a bar chart. In some embodiments, the processing device 120 may generate a motion projection of the target object based on the motion monitoring signals and generate movement probability based on the motion projection of the target object and reference information. The motion projection is a one-dimensional projection or a multi-dimensional projection of the motion monitoring signals.

FIG. 8 is a flowchart illustrating an exemplary process for medical imaging according to some embodiments of the present disclosure. In some embodiments, process 800 may be executed by the medical imaging system 100. For example, process 800 may be implemented as a set of instructions (e.g., an application) stored in a storage device (e.g., the storage device 130), and the processing device 120 (e.g., one or more modules illustrated in FIG. 2) may execute the set of instructions and may accordingly be directed to perform the process 800.

In 810, the processing device 120 (e.g., the obtaining module 210) may obtain a scanning duration of the scanning process.

In some embodiments, the processing device 120 may obtain the scanning duration of the scanning process based on user input. In some embodiments, the processing device 120 may obtain the scanning duration of the scanning process from an imaging plan determined in advance. In some embodiments, different imaging sequences correspond to different scanning durations, and accordingly, the processing device 120 may obtain the scanning duration of the scanning process based on the imaging sequence of the scanning process. In some embodiments, different scan sites correspond to different scanning durations, and accordingly, the processing device 120 may obtain the scanning duration of the scanning process based on the scan site of the target object.

In 810, the processing device 120 (e.g., the imaging module 230) may determine whether the scanning duration is larger than or equal to a preset time threshold.

In some embodiments, the preset time threshold may be related to an imaging speed requirement and/or an imaging quality (e.g., clarity) requirement. Scanning processes with high imaging quality requirements generally last for a long time, so the probability of movement of the target object is high. Therefore, for scanning processes that do not require high imaging quality, the processing device 120 may set the preset time threshold to a higher value; for scanning processes that require high imaging quality, the processing device 120 may set the preset time threshold to a lower value. Scanning processes that require fast imaging generally have a shorter duration and therefore a lower probability of target object movement. Therefore, for scanning processes that require fast imaging, the processing device 120 may set the preset time threshold to a higher value; and for scanning processes that do not require fast imaging, the processing device 120 may set the preset time threshold to a lower value.

In some embodiments, the processing device 120 may determine the preset time threshold based on historical imaging data of the target object. For target objects with low motion probability in historical imaging, the processing device 120 may set the preset time threshold to a higher value; and for target objects with high motion probability in historical imaging, the processing device 120 may set the preset time threshold to a lower value.

In response to the scanning duration being larger than or equal to the preset time threshold, the processing device 120 may perform the obtaining and displaying of motion state information of the target object, that is perform operations 830-850. In response to the scanning duration being less than the preset time threshold, the processing device 120 may perform the scanning process directly to obtain the medical image of the target object.

According to the embodiments of the present disclosure, the processing device 120 determines whether to enable the obtaining and displaying of the motion status information of the target object based on whether the scan duration is greater than the preset time threshold, so that the obtaining and displaying of the motion status information is only enabled for certain scanning processes or certain target objects, thereby improving the system running speed and improving imaging efficiency.

In 830, the processing device 120 (e.g., the obtaining module 210) may obtain the motion state information of an ROI of the target object during a scanning process of the target object. The scanning process includes multiple scan phases that are scanned sequentially.

In 840, during the scanning process, the processing device 120 (e.g., the displaying module 220) may display the motion state information corresponding to at least one of multiple scan phases that has been scanned.

In 850, after the scanning process is completed, the processing device 120 (e.g., the displaying module 220) may display the motion state information corresponding to the multiple scan phases and the medical image of the target object obtained in the scanning process.

More descriptions of the obtaining and displaying of motion state information may be found elsewhere in the present disclosure (e.g., FIG. 3 and the descriptions thereof).

In 860, the processing device 120 (e.g., the storing module 240) may store, based on a preset storage rule, the medical image of the target object and the motion state information corresponding to the multiple scan phases.

In some embodiments, the preset storage rule includes associating and storing the medical image of the target object and the motion state information corresponding to the medical image. For example, the processing device 120 stores, in a first preset region of at least one storage device (e.g., the storage device 130), the medical image of the target object and the motion state information corresponding to the multiple scan phases. Specifically, the medical image and the motion state information corresponding to the same scanning process may be stored in the same preset region of the at least one storage device.

In some embodiments, the preset storage rule includes storing, in the first preset region of the at least one storage device, the medical image of the target object, the motion state information corresponding to the multiple scan phases, and a second visual chart of the motion state information corresponding to the multiple scan phases. Specifically, the medical image, the motion state information, and the second visual chart corresponding to the same scanning process may be stored in the same preset region of the at least one storage device.

By associating and storing the medical image and the motion state information corresponding to the same scanning process, the retrieval rate of the medical image and the motion state information can be improved, thereby improving the operation of the system. By associating and storing the medical image, the motion state information, and the second visual chart corresponding to the same scanning process, the retrieval rate of the medical image, the motion state information, and the second visual chart can be improved, thereby improving the operation of the system.

In some embodiments, the preset storage rule includes storing the medical image of the target object in the first preset region of the at least one storage device, and storing the motion state information corresponding to the multiple scan phases in a second preset region of the at least one storage device. Specifically, the medical image and the motion state information corresponding to the same scanning process may be stored in different preset regions of the at least one storage device.

In some embodiments, the preset storage rule includes storing the medical image of the target object in the first preset region of the at least one storage device, and storing the motion state information corresponding to the multiple scan phases and a second visual chart of the motion state information corresponding to the multiple scan phases in a second preset region of the at least one storage device. Specifically, the medical image, the motion state information, and the second visual chart corresponding to the same scanning process may be stored in different preset regions of the at least one storage device.

By separately storing the medical image and the motion state information corresponding to the same scanning process, the memory requirements for the corresponding preset region are reduced. By separately storing the medical image, the motion state information, and the second visual chart corresponding to the same scanning process, the memory requirements for the corresponding preset region are reduced.

In some embodiments, the preset storage rule includes storing, in a same preset region or different preset regions of the at least one storage device, motion state information corresponding to the multiple scan phases and second visual charts obtained in different scanning processes.

By storing the motion state information and the second visual charts obtained in different scanning processes in the same preset region of the at least one storage device, partitioning requirements for the at least one storage device are reduced. By storing the motion state information and the second visual charts obtained in different scanning processes in the different preset regions of the at least one storage device, the retrieval rate of the motion state information and the second visual charts can be improved, thereby improving the operation of the system.

It should be noted that the medical image, the motion state information, and the second visual chart may be stored in a form of Dicom to facilitate subsequent loading and calling.

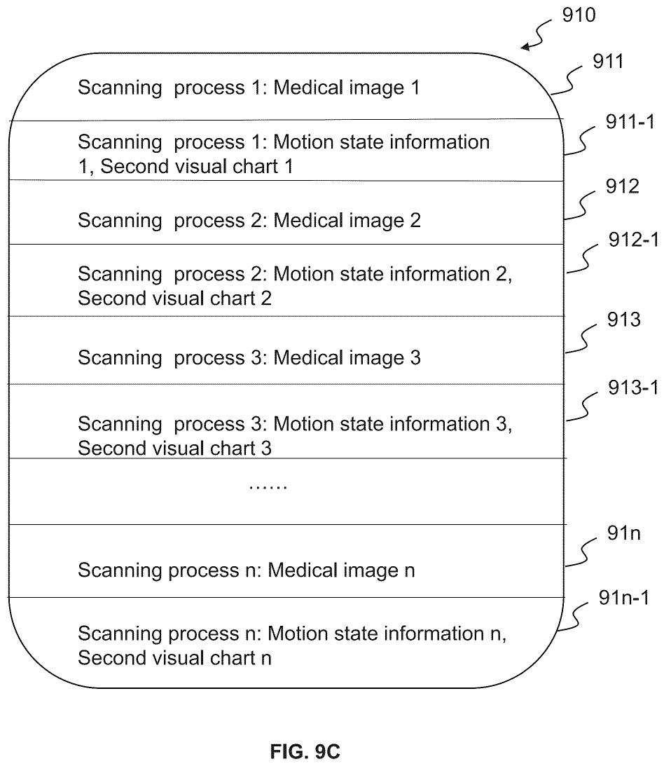

FIGS. 9A-9C are schematic diagrams illustrating exemplary storing of medical images and motion state information according to some embodiments of the present disclosure.

As shown in FIG. 9A, a storage device 910 includes a preset region 911, a preset region 912, a preset region 913, . . . , and a preset region 91n each of which may store a medical image, motion state information, and a second visual chart corresponding to the same scanning process. For example, a medical image 1, motion state information 1, and a second visual chart 1 corresponding to a scanning process 1 are stored in the preset region 911 of the storage device 910; a medical image 2, motion state information 2, and a second visual chart 2 corresponding to a scanning process 2 are stored in the preset region 912 of the storage device 910; a medical image 3, motion state information 3, and a second visual chart 3 corresponding to a scanning process 3 are stored in the preset region 913 of the storage device 910; . . . ; a medical image n, motion state information n, and a second visual chart n corresponding to a scanning process n are stored in the preset region 91n of the storage device 910.

As shown in FIG. 9B, the preset region 911, the preset region 912, the preset region 913, . . . , and the preset region 91n each of which may store a medical image corresponding to a scanning process. For example, the preset region 911 may store the medical image 1; the preset region 912 may store the medical image 2; the preset region 913 may store the medical image 3; and the preset region 91n may store the medical image n. The storage device 910 further includes a preset region 920 that can store the motion state information and the second visual charts corresponding to all scanning processes (e.g., the motion state information 1 and the second visual chart 1 corresponding to the scanning process 1, the motion state information 2 and the second visual chart 2 corresponding to the scanning process 2, the motion state information 3 and the second visual chart 3 corresponding to the scanning process 3, the motion state information n and the second visual chart n corresponding to the scanning process n).

As shown in FIG. 9C, the preset region 911, the preset region 912, the preset region 913, . . . , and the preset region 91n each of which may store a medical image corresponding to a scanning process. For example, the preset region 911 may store the medical image 1; the preset region 912 may store the medical image 2; the preset region 913 may store the medical image 3; and the preset region 91n may store the medical image n. The storage device 910 further includes a preset region 911-1, a preset region 912-1, a preset region 913-1, . . . , and a preset region 91n-1. The preset region 911-1 may store the motion state information 1 and the second visual chart 1 corresponding to the scanning process 1. The preset region 912-1 may store the motion state information 2 and the second visual chart 2 corresponding to the scanning process 2. The preset region 913-1 may store the motion state information 3 and the second visual chart 3 corresponding to the scanning process 3. The preset region 91n-1 may store the motion state information n and the second visual chart n corresponding to the scanning process n.

The operations of the illustrated processes 300 and 800 presented above are intended to be illustrative. In some embodiments, a process may be accomplished with one or more additional operations not described, and/or without one or more of the operations discussed. Additionally, the order in which the operations of a process described above is not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” may mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.), or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C #, VB. NET, Python, or the like, conventional procedural programming languages, such as the “C” programming language, Visual Basic, Fortran 2103, Perl, COBOL 2102, PHP, ABAP, dynamic programming languages such as Python, Ruby, and Groovy, or other programming languages. The program code may execute entirely on the user's computer, partly on the user's computer, as a stand-alone software package, partly on the user's computer, and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A method implemented on a medical imaging device including at least one processor and at least one storage device, the method comprising:
obtaining motion state information of a region of interest (ROI) of a target object during a scanning process of the target object, wherein the scanning process includes multiple scan phases that are scanned sequentially, wherein the motion state information includes at least one of a movement probability or a movement level representing a position change of the target object in each scan phase; and
performing at least one of:
during the scanning process, displaying the motion state information corresponding to at least one of multiple scan phases that has been scanned; or
after the scanning process is completed, displaying the motion state information corresponding to the multiple scan phases and a medical image of the target object obtained in the scanning process.

2. The method of claim 1, wherein during the scanning process, the displaying the motion state information corresponding to at least one of multiple scan phases that has been scanned includes:
forming, based on an execution order of the at least one of multiple scan phases, a first visual chart of the motion state information corresponding to the at least one of multiple scan phases that has been scanned; and
displaying the first visual chart.

3. The method of claim 2, wherein the displaying the first visual chart includes:
obtaining a preset threshold; and
highlighting elements of the first visual chart corresponding to the motion state information based on the preset threshold.

4. The method of claim 1, wherein after the scanning process is completed, the displaying the motion state information corresponding to the multiple scan phases and a medical image of the target object obtained in the scanning process includes:
displaying, based on a preset display rule in a same display interface, the motion state information corresponding to the multiple scan phases and the medical image of the target object.

5. The method of claim 4, wherein the preset display rule includes at least one of:
displaying the motion state information corresponding to the multiple scan phases in a displaying region on the display interface;
displaying the motion state information exceeding a preset threshold in the displaying region;
forming, based on an execution order of the multiple scan phases, a second visual chart of the motion state information corresponding to the multiple scan phases, and displaying the second visual chart in the displaying region;
displaying the second visual chart in the displaying region, and highlighting elements of the second visual chart corresponding to the motion state information exceeding the preset threshold; or
marking an image region corresponding to the motion state information exceeding the preset threshold in the medical image.

6. The method of claim 5, wherein the displaying region includes a region, in the display interface, not occupied by the medical image, at least one of four corners of the medical image, or a pop-up window of the display interface.

7. The method of claim 5, wherein the first visual chart or the second visual chart includes at least one of a scatter chart, a line chart, or a bar chart.

8. The method of claim 2, wherein the preset threshold is related to a scan site of the ROI of the target object.

9. The method of claim 1, wherein the method further includes:
storing, based on a preset storage rule, the medical image of the target object and the motion state information corresponding to the multiple scan phases.

10. The method of claim 9, wherein the preset storage rule includes storing, in a first preset region of the at least one storage device, the medical image of the target object, the motion state information corresponding to the multiple scan phases, and a second visual chart of the motion state information corresponding to the multiple scan phases.

11. The method of claim 9, wherein the preset storage rule includes storing the medical image of the target object in a first preset region of the at least one storage device, and storing the motion state information corresponding to the multiple scan phases and a second visual chart of the motion state information corresponding to the multiple scan phases in a second preset region of the at least one storage device.

12. The method of claim 9, wherein the preset storage rule includes storing, in a same preset region or different preset regions of the at least one storage device, motion state information corresponding to the multiple scan phases and second visual charts obtained in different scanning processes.

13. The method of claim 1, wherein the obtaining motion state information includes:
obtaining the motion state information by scanning the target object using a motion detection device inserted into the multiple scan phases in the scanning process.

14. A system, comprising:
at least one storage device including a set of instructions; and
at least one processor in communication with the at least one storage device, wherein when executing the set of instructions, the at least one processor causes the system to perform operations including:
obtaining motion state information of a region of interest (ROI) of a target object during a scanning process of the target object, wherein the scanning process includes multiple scan phases that are scanned sequentially, wherein the motion state information represents a motion trend of the ROI of the target object in each scan phase; and
performing at least one of:
during the scanning process, displaying the motion state information corresponding to at least one of multiple scan phases that has been scanned; or
after the scanning process is completed, displaying the motion state information corresponding to the multiple scan phases and a medical image of the target object obtained in the scanning process.

15. (canceled)

16. (canceled)

17. (canceled)

18. (canceled)

19. A method implemented on a medical imaging device including at least one processor and at least one storage device, the method comprising:
obtaining a scanning sequence for scanning a target object, the scanning sequence including multiple scan phases;
for at least one of the multiple scan phases, obtaining motion monitoring signals of the target object by applying a motion detection sequence before the scan phase;
generating motion state information of the target object based on the motion monitoring signals, the motion state information including a movement probability or a movement level representing a position change of the target object in the scan phase; and
controlling a display interface of the medical imaging device to display the movement probability or the movement level.

20. The method of claim 19, wherein
the display interface sequentially displays movement probabilities or movement levels corresponding to the multiple scan phases in a form of a scatter plot, a line chart, or a bar chart, and
the method further inlcudes:
generating a motion projection of the target object based on the motion monitoring signals, the motion projection being a one-dimensional projection or a multi-dimensional projection of the motion monitoring signals; and
generating movement probability based on the motion projection of the target object and reference information.

21. The system of claim 14, wherein during the scanning process, the displaying the motion state information corresponding to at least one of multiple scan intervals that has been scanned includes:
forming, based on an execution order of the at least one of multiple scan intervals, a first visual chart of the motion state information corresponding to the at least one of multiple scan intervals that has been scanned; and
displaying the first visual chart.

22. The system of claim 21, wherein the displaying the first visual chart includes:
obtaining a preset threshold; and
highlighting elements of the first visual chart corresponding to the motion state information based on the preset threshold.

23. The system of claim 14, wherein after the scanning process is completed, the displaying the motion state information corresponding to the multiple scan intervals and a medical image of the target object obtained in the scanning process includes:
displaying, based on a preset display rule in a same display interface, the motion state information corresponding to the multiple scan intervals and the medical image of the target object.

24. The system of claim 23, wherein the preset display rule includes at least one of:
displaying the motion state information corresponding to the multiple scan intervals in a displaying region on the display interface;
displaying the motion state information exceeding a preset threshold in the displaying region;
forming, based on an execution order of the multiple scan intervals, a second visual chart of the motion state information corresponding to the multiple scan intervals, and displaying the second visual chart in the displaying region;
displaying the second visual chart in the displaying region, and highlighting elements of the second visual chart corresponding to the motion state information exceeding the preset threshold; or
marking an image region corresponding to the motion state information exceeding the preset threshold in the medical image.
